(19) United States
(12) Patent Application Publication
(10) Pub. No.: US 2023/0240780 A1
(43) Pub. Date: Aug. 3, 2023

(54) PROTECTION SYSTEMS, ASSEMBLIES, AND DEVICES

(71) Applicant: Mazor Robotics Ltd., Caesarea (IL)

(72) Inventors: Nimrod Dori, Atlit (IL); Dor Kopito, Kibbutz Parod (IL); Jaffar Hleihil, Zichron Yaakov (IL)

(21) Appl. No.: 17/590,928

(22) Filed: Feb. 2, 2022

(51) Int. Cl. A61B 34/00; A61B 34/30; B25J 13/08; B25J 9/16; A61B 34/20

(52) U.S. Cl. CPC A61B 34/71; A61B 34/30; B25J 13/089; B25J 9/1602; A61B 34/20; A61B 2034/2074

(57) ABSTRACT

Protection systems, assemblies, and devices are provided. A protection assembly may be configured to transition an end unit of a robot between a first state and a second state. A signal indicating a breaching state may be received. The protection assembly may transition the end unit from the first state to the second state when the signal is received.

### BACKGROUND

The present disclosure is generally directed to protection systems, assemblies, and devices, and relates more particularly to protection systems, assemblies, and devices for protecting an end unit of a robot.

Surgical robots may assist a surgeon or other medical provider in carrying out a surgical procedure, or may complete one or more surgical procedures autonomously. Providing controllable linked articulating members allows a surgical robot to reach areas of a patient anatomy during various medical procedures.

### BRIEF SUMMARY

Example aspects of the present disclosure include:

A protection system according to at least one embodiment of the present disclosure comprises a protection assembly configured to transition an end unit of a robot between a first state and a second state; a processor; and a memory storing data for processing by the processor, the data, when processed, causes the processor to: receive a signal indicating a breaching state; and cause the protection assembly to transition the end unit from the first state to the second state when the signal is received.

Any of the aspects herein, wherein the end unit comprises a surgical tool or a surgical instrument.

Any of the aspects herein, wherein the breaching state comprises a breach of the end unit from a hard tissue to a soft tissue.

Any of the aspects herein, wherein the first state comprises a working state and the second state comprises a non-working state.

Any of the aspects herein, wherein the protection assembly comprises: a platform configured to receive the end unit; a biasing member configured to bias the end unit to the second state; and a locking mechanism configured to lock the biasing member, wherein when the locking mechanism releases the biasing member, the biasing member transitions the end unit from the first state to the second state.

Any of the aspects herein, wherein the end unit is extended when in the first state and retracted when in the second state.

Any of the aspects herein, wherein causing the protection assembly to transition the end unit from the first state to the second state comprises causing the locking mechanism to release the biasing member.

Any of the aspects herein, wherein the locking mechanism comprises an electro-mechanical device.

Any of the aspects herein, wherein the protection assembly comprises: a sleeve configured to transition between a first sleeve position and a second sleeve position, wherein the sleeve covers the end unit when in the first sleeve position and does not cover the end unit when in the second sleeve position, wherein the second sleeve position correlates to the first state of the end unit and the first sleeve position correlates to the second state of the end unit; a biasing member configured to bias the sleeve to the first sleeve position; and a locking mechanism configured to lock the biasing member in a locked state, wherein the biasing member does not bias the sleeve when in the locked state, and wherein when the locking mechanism releases the biasing member from the locked state, the biasing member transitions the sleeve from the second sleeve position to the first sleeve position.

Any of the aspects herein, wherein the protection assembly further comprises a platform, the sleeve disposed on the platform, wherein the sleeve is extended away from the platform and covers the end unit when in the first sleeve position and is positioned on the platform when in the second sleeve position.

A protection system according to at least one embodiment of the present disclosure comprises a platform configured to support an end unit, the end unit configured to transition between a first state and a second state; a biasing member configured to bias the end unit to the second state; and a locking mechanism configured to lock the biasing member in a locked state, wherein the biasing member does not bias the end unit when in the locked state, and wherein when the locking mechanism releases the biasing member from the locked state, the biasing member transitions the end unit from the first state to the second state.

Any of the aspects herein, wherein the end unit comprises a surgical tool or a surgical instrument.

Any of the aspects herein, wherein the biasing member comprises a spring and the locking mechanism is a solenoid, wherein the spring is compressed when in the locked state. Any of the aspects herein, wherein the end unit is extended when in the first state and retracted when in the second state.

Any of the aspects herein, wherein the platform comprises a first end opposite a second end.

Any of the aspects herein, wherein the end unit is extended away from the first end and past the second end when in the first state and retracted towards the first end when in the second state.

A protection system according to at least one embodiment of the present disclosure comprises a sleeve movable between a first sleeve position and a second sleeve position, wherein the sleeve covers an end unit of a robot when in the first sleeve position and does not cover the end unit when in the second sleeve position; a biasing member configured to bias the sleeve to the first sleeve position; and a locking mechanism configured to lock the biasing member in a locked state, wherein the biasing member does not bias the sleeve when in the locked state, and wherein when the locking mechanism releases the biasing member from the locked state, the biasing member transitions the sleeve from the second sleeve position to the first sleeve position.

Any of the aspects herein, wherein the end unit comprises a surgical tool or a surgical instrument.

Any of the aspects herein, wherein the biasing member comprises a spring.

Any of the aspects herein, further comprising a platform configured to receive the sleeve, wherein the sleeve is extended away from the platform and covers the end unit when in the first sleeve position and is positioned on the platform when in the second sleeve position.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a schematic diagram of an end unit in a first state according to at least one embodiment of the present disclosure;

FIG. 2B is a schematic diagram of an end unit in a second state according to at least one embodiment of the present disclosure;

FIG. 3A is a schematic diagram of an end unit in a first state according to at least one embodiment of the present disclosure;

FIG. 3B is a schematic diagram of an end unit in a second state according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During a robotic surgical procedure or a robot assisted surgical procedure such as, for example, a spinal procedure, a surgical tool operated by a robot may breach hard tissue such as bone to soft tissue. When such breach is detected, stopping an inward motion of the surgical tool and reversing the motion includes decelerating the surgical tool and robotic arm and accelerating the large mass in the opposite direction. Such motion may be slow to execute. Another latency may be introduced from sending commands from a controller to each motor in the robotic arm.

At least one embodiment of the present disclosure provides for a spring-loaded rail that may be, for example, 2-3 cm in length, that is installed at an end-unit of a robotic arm. The rail may be aligned with a trajectory of the end unit. The rail may be locked in place by an electro-mechanical locking device, while the spring is compressed. When an emergency retreat is triggered, the rail lock may be released, and the spring may push the end-unit outwards on the rail, which distances a tip of the end unit from, for example, sensitive soft tissue.

In an alternative embodiment of the present disclosure, the spring may push down a protective sleeve around the end unit to protect the patient from the end unit and/or to protect the end unit from damage.

Embodiments of the present disclosure provide technical solutions to one or more of the problems of (1) preventing an end unit from undesirably contacting a patient, (2) preventing an end unit from breaching hard tissue to soft tissue, (3) providing a safety mechanism for autonomous or semi-autonomous surgical robots, and (4) increasing patient and surgical team safety.

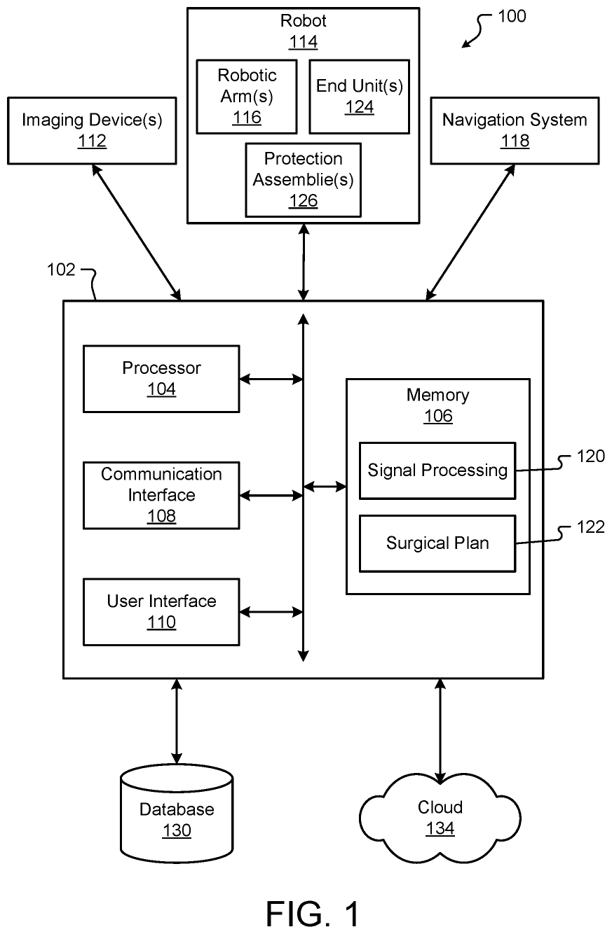

Turning first to FIG. 1, a block diagram of a system 100 according to at least one embodiment of the present disclosure is shown. The system 100 may be used to protect an end unit such as an end unit 124 of a robot such as a robot 114 using a protection assembly such as a protection assembly 126 and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable signal processing 120.

The signal processing 120 enables the processor 104 to process signal data (received from for example, a protection assembly 126, the computing device 102, or any component of the system 100 or any component outside of the system 100) for the purpose of, for example, identifying one or more states of end unit 124. The states may comprise, for example, a working state, a breaching state, a non-working or standby state, or any other state. The working state may correlate to whether the end unit 124 is in a working state and ready for use. For example, the working state may indicate that power is received by the end unit 124.

The breaching state may indicate that the end unit 124 has breached or may breach, for example, hard tissue such as a bone. When such breaching state is triggered, in some embodiments, the end unit 124 is retracted away from, for example, the hard tissue. In other embodiments, when the breaching state is triggered, a sleeve such as a sleeve 330 is moved downward to cover the end unit 124. The standby state may indicate that the end unit 124 is not in use. In the standby state, the end unit 124 may be retracted or may be covered by the sleeve 330 to protect the end unit 124 from damage and/or to prevent damage by the end unit 124 to, for example, a patient or a surgical team.

The content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The memory 106 may also store a surgical plan 122. The surgical plan 122 may comprise, for example, one or more steps for performing a surgical procedure. For example, the surgical plan 122 may include steps for activating the end unit 124 to put the end unit 124 in the working condition and steps for deactivating the end unit 124 to put the end unit 124 in the standby condition. The surgical plan 122 may also comprise one or more thresholds to be monitored during a surgical procedure. In some embodiments, the surgical procedure may be a spinal procedure (e.g., a spinal alignment, installing implants, osteotomy, fusion, and/or any other spinal procedure). The surgical plan 122 may also be stored in the database 130.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position, for example, the protection assembly 126 and the end unit 124 at one or more precise position(s) and orientation(s), and/or to return the protection assembly 126 and the end unit 124 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the protection assembly 126 and the end unit 124. In embodiments where two protection assemblies 126 and end units 124 are used, one robotic arm 116 may hold one set, and another robotic arm 116 may hold another set. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, the protection assembly 126, the end unit 124, a surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, the protection assembly 126, the end unit 124 or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., the protection assembly 126 and the end unit 124) and the system can operate without the use of the robot 114 (e.g., with the surgeon manually manipulating the protection assembly 126 and the end unit 124 and/or one or more surgical tools, based on information and/or instructions generated by the navigation system 118, for example).

The robot 114 may also comprise one or more protection assemblies 126 for transitioning an end unit 124 between a first state and a second state. It will be appreciated that the protection assembly 126 may move the end unit 124 to any number of states. The end unit 124 may comprise, for example, a surgical instrument and/or a surgical tool. In some embodiments the end unit 124 is extended from a platform 204, 304 when in the first state and retracted to the platform 204, 304 when in the second state. In other embodiments, the end unit 124 is uncovered when in the first state and covered by, for example, a sleeve such as the sleeve 330, when in the second state. The protection assembly 126 may be oriented by, for example, the robotic arm 116. The protection assembly 126 (and thus the end unit 124) may be positioned at an end of the robotic arm 116 or on any portion of the robot 114 and/or the robotic arm 116.

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the protection assembly 126, the end unit 124, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans 122 (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the method 400 described herein. The system 100 or similar systems may also be used for other purposes.

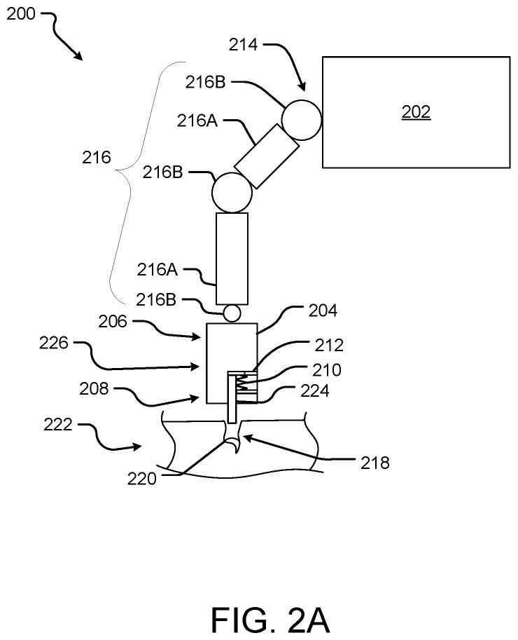

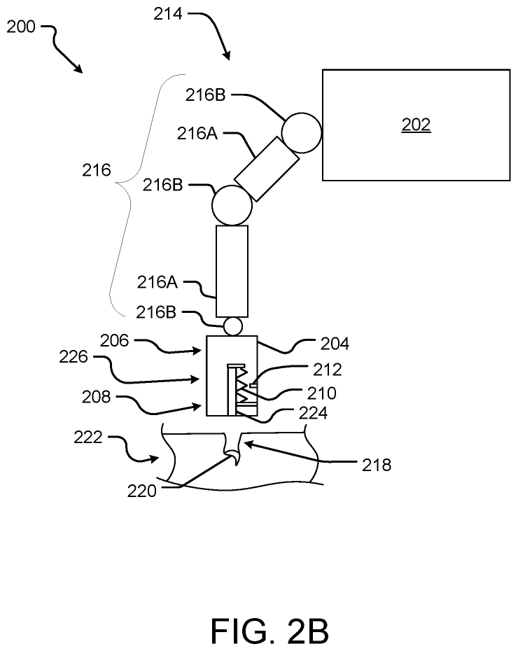

Turning to FIGS. 2A and 2B, a diagram of a system 200 comprising an end unit 224 in a first state and a diagram of the system 200 comprising the end unit 224 in a second state are respectively illustrated. The system 200 comprises a protection assembly 226, which may be the same as or similar to the protection assembly 126 described above. The system 200 also comprises a robot 214, which may be the same as or similar to the robot 114 described above. Systems according to other embodiments of the present disclosure may comprise more or fewer components than the system 200. For example, the system 200 may comprise a navigation system, a computing device, etc.

As illustrated, the robot 214 comprises a robotic arm 216 (which may comprise one or more members 216A connected by one or more joints 216B) extending from a base 202. In other embodiments, the robot 214 may comprise one robotic arm or two or more robotic arms. The base 202 may be stationary or movable. In the illustrated embodiment, the protection assembly 226 is disposed at an end of the robotic arm 216. It will be appreciated that in other embodiments, an imaging device such as the imaging device 112, one or more surgical tools, one or more surgical instruments, or any other component may be disposed at the end of the robotic arm 216 or on any portion of the robotic arm 216 or the robot 214. The robotic arm 216 is operable to execute one or more planned movements and/or procedures autonomously and/or based on input from a surgeon or user. The robotic arm 216 may also orient and/or operate the protection assembly 226.

In at least one embodiment, the protection assembly 226 comprises a platform 204 configured to support an end unit 224. The platform 204 comprises a first end 206 opposite the second end 208. The end unit 224 may be the same as or similar to the end unit 124. As previously described, the end unit 224 may be transitioned between a first state in which the end unit 224 is extended from the platform 204—as shown in FIG. 2A—and a second state in which the end unit 224 is retracted to the platform 204—as shown in FIG. 2B. When the end unit 224 is in the first state, the end unit 224 may be operable to perform a surgical step. For example, the end unit 224 may comprise a surgical drill and the surgical step may comprise drilling an anatomical element such as bone.

The protection assembly 226 may also comprise a biasing member 210 configured to bias the end unit 224 to the second state and a locking mechanism 212 configured to lock the biasing member 210. The locking mechanism 212 may lock the biasing member 210 so that the end unit 224 is operable in the first state. When the locking mechanism 212 releases the biasing member 210, the biasing member 210 may transition the end unit 224 from the first state to the second state. In some embodiments, the biasing member 210 may be, for example, a spring coupled to the end unit 224. It will be appreciated that in other embodiments, the biasing member 210 may be, for example, a motor configured to move the end unit 224 between the first state and the second state. In some embodiments, the locking mechanism 212 may be an electro-mechanical locking mechanism such as, for example, a solenoid and a lever. The solenoid may hold the lever to lock the biasing member 210 (when the biasing member 210 comprises, for example, a spring) in a locked stated.

In some embodiments, the platform 204 may comprise a rail to which the end unit 224 is coupled to. The end unit 224 may be movable between the first state and the second state on the rail. The rail, in some embodiments, may be, for example 2-3 cm in length. In other embodiments, the rail may be less than 2 cm or greater than 3 cm in length. The rail may also be aligned with a trajectory of the end unit 224 such that when the end unit 224 is retracted the end unit 224 reverses direction and moves in an opposite direction from an initial direction along the trajectory. In some embodiments, the platform may comprise a housing and the end unit 224 may be retracted from and into the housing.

In embodiments where the biasing member 210 is a spring, the spring may be locked in a compressed state by the locking mechanism 212 so that the end unit 224 is operable in the first state. When the locking mechanism 212 releases the spring, the spring expands and moves the end unit 224 along the rail to retract the end unit 224 towards the platform 204. It will be appreciated that in other embodiments, the spring may be locked in an expanded state by the locking mechanism 212 so that the end unit 224 is operable in the first state. In such embodiments, when the locking mechanism 212 releases the spring, the spring may compress and move the end unit 224 towards the platform 204.

As shown in FIG. 2A, the end unit 224 may be in the first state and extended towards a surgical site. The biasing member 210 may be locked by the locking mechanism 212 in a locked and/or compressed state such that the end unit 224. In the illustrated embodiment, the surgical site comprises an incision 218 formed on a patient 222 to access hard tissue 220 such as bone. The end unit 224 may comprise, for example, a surgical drill to drill the hard tissue 220. During a surgical procedure (such as, for example, drilling the hard tissue 220), the surgical drill may be monitored to ensure that the surgical drill does not breach the hard tissue. Breaching the hard tissue by the surgical drill may harm surrounding soft tissue, and thus, it is desirable to remove the surgical drill from the surgical site if a breach occurs or is imminent. The surgical drill may be monitored by, for example, an imaging device such as the imaging device 112.

As shown in FIG. 2B, the locking mechanism 212 may release the biasing member 210, thereby transitioning the end unit 224 from the first state to the second state when a breach is detected and/or when a threshold is met or exceeded. The threshold may be, for example, a thickness threshold. The thickness threshold may be, for example, a distance threshold measured from a tip of the surgical drill to a boundary of soft tissue, which may correlate to a thickness of the hard tissue. In other words, the thickness of the hard tissue may be monitored (using, for example, imaging such as ultrasound imaging) and when the thickness of the hard tissue meets or exceeds a minimum thickness threshold, the locking mechanism 212 may release the biasing member 210 to transition the end unit 224 from the first state to the second state. Such minimum thickness threshold may prevent breaching of the hard tissue to the soft tissue by the end unit 224. In some embodiments, the end unit 224 may be transitioned from the first state to the second state when the surgical procedure is completed to protect a surgical site from the end unit 224 and/or the protect the end unit 224 from damage. After the end unit 224 is transitioned to the second state, the end unit 224 may be transitioned to the first state based on, for example, input from a surgeon, the measured distance no longer meeting or exceeding the distance threshold, and/or based on a surgical step received from a surgical plan such as the surgical plan 122.

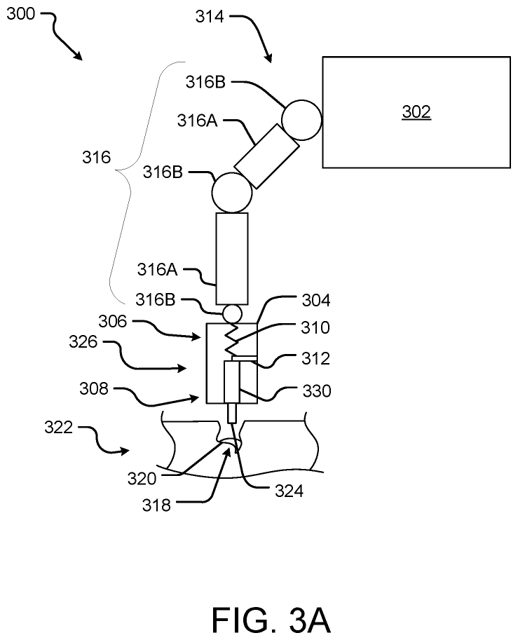

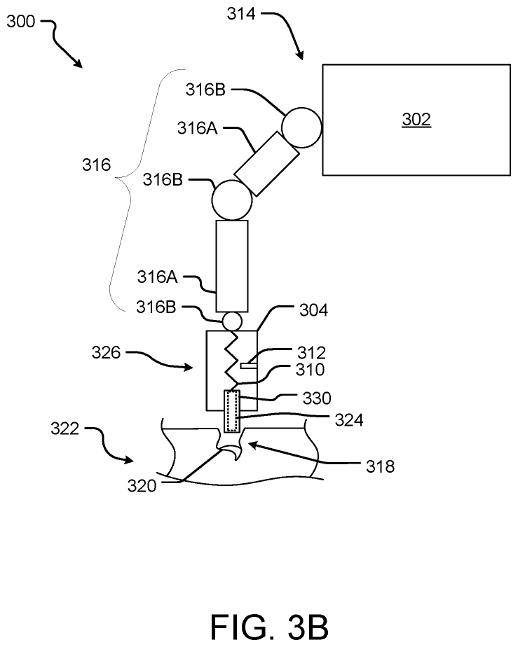

Turning to FIGS. 3A and 3B, a diagram of a system 300 comprising an end unit 324 in a first state and a diagram of the system 300 comprising the end unit 324 in a second state are respectively illustrated. The system 300 comprises a protection assembly 326, which may be the same as or similar to the protection assembly 126, 226 described above. The system 300 also comprises a robot 314, which may be the same as or similar to the robot 114, 214 described above. Systems according to other embodiments of the present disclosure may comprise more or fewer components than the system 300. For example, the system 300 may comprise a navigation system, a computing device, etc.

As illustrated and similarly described above, the robot 314 comprises a robotic arm 316 (which may comprise one or more members 316A connected by one or more joints 316B) extending from a base 302. In other embodiments, the robot 314 may include one robotic arm or two or more robotic arms. The base 302 may be stationary or movable. In the illustrated embodiment, the protection assembly 326 is disposed at an end of the robotic arm 316. It will be appreciated that in other embodiments, an imaging device such as the imaging device 112, one or more surgical tools, one or more surgical instruments, or any other component may be disposed at the end of the robotic arm 316 or on any portion of the robotic arm 316 or the robot 314. The robotic arm 316 is operable to execute one or more planned movements and/or procedures autonomously and/or based on input from a surgeon or user.

In at least one embodiment, the protection assembly 326 comprises a platform 304 configured to support an end unit 324 and a sleeve 330. The end unit 324 may be the same as or similar to the end unit 124, 224. In some embodiments, the end unit 324 may be fixed to the platform 304. In other embodiments, the end unit 324 may be moveable. For example, the end unit 324 may be extended from the platform 304 or retracted to the platform 304. The protection assembly 326 may also comprise the sleeve 330 configured to transition between a first sleeve position and a second sleeve position. The sleeve 330 covers the end unit 324 when in the first sleeve position and does not cover the end unit 324 when in the second sleeve position. The first sleeve position also correlates to the second state of the end unit 324 (e.g., when the end unit 324 is in a non-working or standby state) and the second sleeve position correlates to the first state of the end unit 324 (e.g., when the end unit 324 is in a working state).

The protection assembly 326 may also comprise a biasing member 310 configured to bias the sleeve 330 to the first sleeve position and a locking mechanism 312 configured to lock the biasing member 310. The locking mechanism 312 may lock the biasing member 310 so that the sleeve 330 is in the second sleeve position and the end unit 324 is operable in the first state. When the locking mechanism 312 releases the biasing member 310, the biasing member 310 may transition the sleeve 330 from the second sleeve position to the first sleeve position. In some embodiments, the biasing member 310 may be, for example, a spring coupled to the sleeve 330. It will be appreciated that in other embodiments, the biasing member 310 may be, for example, a motor configured to move the sleeve 330 between the first sleeve position and the second sleeve position. In some embodiments, the locking mechanism 312 may be an electro-mechanical locking mechanism such as, for example, a solenoid and a lever. The solenoid may hold the lever to lock the biasing member 310 (when the biasing member 310 comprises, for example, a spring) in a locked stated.

In some embodiments, the platform 304 may comprise a rail to which the sleeve 330 is coupled to and is movable between the first sleeve position and the second sleeve position on the rail. In embodiments where the biasing member is a spring, the spring may be locked in a compressed state by the locking mechanism 312 so that the end unit 324 is operable in the first state. When the locking mechanism 312 releases the spring, the spring expands and transitions the sleeve 330 from the second sleeve position to the first sleeve position.

As shown in FIG. 3A, the sleeve 330 is in the second sleeve position and does not cover the end unit 324. The biasing member 310 may be locked by the locking mechanism 312 in a locked and/or compressed state such that the end unit 324 may be uncovered operable in the first state. In the illustrated embodiment, the surgical site comprises an incision 318 formed on a patient 322 to access hard tissue 320 such as bone. The end unit 324 may comprise, for example, a surgical drill to drill the hard tissue 320. During a surgical procedure (such as, for example, drilling the hard tissue 320), the surgical drill may be monitored to ensure that the surgical drill does not breach the hard tissue. As previously described, breaching the hard tissue by the surgical drill may harm surrounding soft tissue, and thus, it is desirable to remove the surgical drill from the surgical site if a breach occurs or is imminent. The surgical drill may be monitored by, for example, an imaging device such as the imaging device 112.

As shown in FIG. 3B, the locking mechanism 312 may release the biasing member 310, thereby transitioning the sleeve 330 from the second sleeve position to the first sleeve position when a breach is detected and/or when a threshold is met or exceeded. As shown, the end unit 324 is shown in dashed line for clarity, as the end unit 324 may be covered by the sleeve 330 when the sleeve 330 is in the first sleeve position (and the end unit 324 is in the second state). The threshold may be, for example, a distance threshold measured from a tip of the surgical drill to soft tissue, which may correlate to a thickness of the hard tissue. In some embodiments, the sleeve 330 may be transitioned from the second sleeve position to the first sleeve position when the surgical procedure is completed to protect a surgical site from the end unit 324 and/or the protect the end unit 324 from damage. After the sleeve 330 is transitioned to the first sleeve position, the sleeve 330 may be transitioned to the second sleeve position based on, for example, input from a surgeon, the measured distance no longer meeting or exceeding the distance threshold, and/or based on a surgical step received from a surgical plan such as the surgical plan 122.

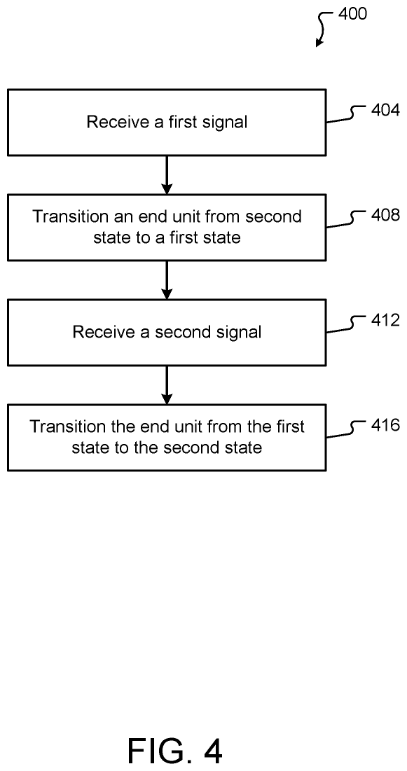

FIG. 4 depicts a method 400 that may be used, for example, for transitioning an end unit between a first state and a second state using a protection assembly, system, and/or device.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as a signal processing 120.

The method 400 comprises receiving a first signal (step 404). The first signal may be received from a protection assembly such as the protection assembly 126, 226, 326, a computing device such as the computing device 102, a navigation system such as the navigation system 118, any component of a system such as the system 100, or any component outside of the system. The protection assembly may be configured to transition an end unit such as the end unit 124, 224, 324 between a first state and a second state. In some embodiments, the end unit is extended from a platform such as the platform 204, 304 when in the first state and the end unit is retracted towards the platform when in the second state. More specifically, in some embodiments, the protection assembly may comprise a biasing member such as the biasing member 210 configured to bias the end unit to the second state and a locking mechanism such as the locking mechanism 212 configured to lock the biasing member in a locked state in which the end unit is held in the first state. When the locking mechanism releases the biasing member from the locked state, the biasing member transitions the end unit from the first state to the second state.

In other embodiments, the protection assembly may comprise a sleeve such as the sleeve 330, a biasing member such as the biasing member 310 configured to bias the sleeve to a first sleeve position, and a locking mechanism such as the locking mechanism 312 configured to lock the biasing member in a locked state in which the sleeve is held in the second sleeve position. In such embodiments, the first sleeve position also correlates to the second state of the end unit (e.g., when the end unit is in a non-working or standby state) and the second sleeve position correlates to the first state of the end unit (e.g., when the end unit is in a working state).

The first signal may be received by a processor such as the processor 104. The processor may use, for example, a signal processing such as the signal processing 120 to process the signal data received for the purpose of identifying one or more states of the end unit. In some embodiments, the first signal may indicate a working state. In other embodiments, the first signal may indicate any one or more states such as, for example, a retracting state or a standby state. The working condition may indicate that the end unit is in condition for use. For example, the working condition may indicate that power is received by the end unit of the protection assembly.

The method 400 also comprises transitioning the end unit from a first state to a second state (step 408). The protection assembly may transition the end unit from the second state to the first state based on receiving the first signal indicating that the end unit is in a working state.

In some embodiments, transitioning the end unit from the first state to the second state comprises causing the locking mechanism to release the biasing member such that the biasing member moves the end unit from the first state to the second state. For example, in some embodiments, the biasing member may comprise a spring coupled to the end unit and the locking mechanism may lock the spring in a compressed state. When the locking mechanism releases the spring, the spring may expand and move the end unit away from a surgical site and towards the platform.

In other embodiments, wherein the protection assembly comprises a sleeve, transitioning the end unit from the first state to the second state comprises causing the locking mechanism to release the biasing member such that the biasing member moves the sleeve from the second sleeve position to the first sleeve position, thereby covering the end unit. For example, in some embodiments, the biasing member may be a spring coupled to the sleeve and the locking mechanism may lock the spring in a compressed state. When the locking mechanism releases the spring, the spring may expand and move the sleeve to the end unit, thereby covering the end unit.

The method 400 also comprises receiving a second signal (step 412). The step 412 may be the same as or similar to the step 404 described above. The second signal may indicate that the end unit is in a breaching state or a non-working or standby state The breaching state may indicate that the end unit has breached or may breach, for example, a hard tissue such as a bone. When such breaching state is triggered, in some embodiments, the end unit is retracted away from, for example, a surgical site. In other embodiments, when the breaching state is triggered, the sleeve is transitioned downward to cover the end unit. The standby state may indicate that the end unit is not in use, for example, at the end of a surgical procedure and/or operation. The standby condition may be activated by, for example, a user such as a surgeon or other medical provider. The standby condition may also be automatically activated by, for example, the processor executing a step of a surgical plan such as the surgical plan 122. In the standby state, the end unit may be retracted or may be covered by the sleeve to protect the end unit from damage and/or to prevent damage from the end unit to, for example, a patient or a surgical team.

The breaching state may be triggered by, for example, when a breach is detected and/or when a threshold is met or exceeded. The threshold may be, for example, a distance threshold measured from a tip of the end unit to soft tissue, which may correlate to a thickness of hard tissue extending between the end unit and the soft tissue. The distance threshold may be determined automatically using artificial intelligence and training data (e.g., historical cases) in some embodiments. In other embodiments, the distance threshold may be or comprise, or be based on, surgeon input received via the user interface. In further embodiments, the distance threshold may be determined automatically using artificial intelligence, and may thereafter be reviewed and approved (or modified) by a surgeon or other user.

The method 400 also comprises transitioning the end unit from the first state to the second state (step 416). The step 416 may be the same as or similar to the step 408 described above with respect to moving the end unit from the first state to the second state. Moving the end unit from the first state to the second state may be based on receiving the second signal indicating the breaching state and/or the non-working or standby state.

In some embodiments, when a distance measured between an end of the end unit and soft tissue meets or exceeds a distance threshold, the protection assembly may move the end unit from the first state to the second state and may also generate a notification to a user such as a surgeon or other medical provide. The notification may alert the user that the measured distance has met or exceeded the distance threshold. The notification may be, for example, an audible sound and/or displayed on a user interface such as the user interface 110.

It will be appreciated that the steps 404-408 and the steps 412-416 may be performed in any order and may be repeated. For example, the steps 412-416 may occur when a measured distance between an end of an end unit and soft tissue meets or exceeds a distance threshold and the steps 404-408 may then occur when the distance does not meet or exceed the distance threshold. The steps 412-416 may repeat when the measured distance meets or exceeds the distance threshold.

In some embodiments, it will be appreciated that the end unit may be held in the first state when the first signal is being received and the end unit may transition from the first state to the second state when the first signal is not received. In other words, in some instances, the end unit may be held in the first state so long as the first signal is continuously received. When the first signal is not received (whether due to, for example, power loss or otherwise), the end unit may automatically transition to the second state.

The present disclosure encompasses embodiments of the method 400 that comprise more or fewer steps than those described above, and/or one or more steps that are different than the steps described above.

As noted above, the present disclosure encompasses methods with fewer than all of the steps identified in FIG. 4 (and the corresponding description of the method 400), as well as methods that include additional steps beyond those identified in FIG. 4 (and the corresponding description of the method 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A protection system comprising:
a protection assembly configured to transition an end unit of a robot between a first state and a second state;
a processor; and
a memory storing data for processing by the processor, the data, when processed, causes the processor to:
receive a signal indicating a breaching state; and
cause the protection assembly to transition the end unit from the first state to the second state when the signal is received.

2. The protection system of claim 1, wherein the end unit comprises a surgical tool or a surgical instrument.

3. The protection system of claim 1, wherein the breaching state comprises a breach of the end unit from a hard tissue to a soft tissue.

4. The protection system of claim 1, wherein the first state comprises a working state and the second state comprises a non-working state.

5. The protection system of claim 1, wherein the protection assembly comprises:
a platform configured to receive the end unit;
a biasing member configured to bias the end unit to the second state; and
a locking mechanism configured to lock the biasing member, wherein when the locking mechanism releases the biasing member, the biasing member transitions the end unit from the first state to the second state.

6. The protection system of claim 5, wherein the end unit is extended when in the first state and retracted when in the second state.

7. The protection system of claim 5, wherein causing the protection assembly to transition the end unit from the first state to the second state comprises causing the locking mechanism to release the biasing member.

8. The protection system of claim 5, wherein the locking mechanism comprises an electro-mechanical device.

9. The protection system of claim 1, wherein the protection assembly comprises:
a sleeve configured to transition between a first sleeve position and a second sleeve position, wherein the sleeve covers the end unit when in the first sleeve position and does not cover the end unit when in the second sleeve position, wherein the second sleeve position correlates to the first state of the end unit and the first sleeve position correlates to the second state of the end unit;
a biasing member configured to bias the sleeve to the first sleeve position; and
a locking mechanism configured to lock the biasing member in a locked state, wherein the biasing member does not bias the sleeve when in the locked state, and wherein when the locking mechanism releases the biasing member from the locked state, the biasing member transitions the sleeve from the second sleeve position to the first sleeve position.

10. The protection system of claim 9, wherein the protection assembly further comprises a platform, the sleeve disposed on the platform, wherein the sleeve is extended away from the platform and covers the end unit when in the first sleeve position and is positioned on the platform when in the second sleeve position.

11. A protection assembly comprising:
a platform configured to support an end unit, the end unit configured to transition between a first state and a second state;
a biasing member configured to bias the end unit to the second state; and
a locking mechanism configured to lock the biasing member in a locked state, wherein the biasing member does not bias the end unit when in the locked state, and wherein when the locking mechanism releases the biasing member from the locked state, the biasing member transitions the end unit from the first state to the second state.

12. The protection assembly of claim 11, wherein the end unit comprises a surgical tool or a surgical instrument.

13. The protection assembly of claim 11, wherein the biasing member comprises a spring and the locking mechanism is a solenoid, wherein the spring is compressed when in the locked state.

14. The protection assembly of claim 11, wherein the end unit is extended when in the first state and retracted when in the second state.

15. The protection assembly of claim 11, wherein the platform comprises a first end opposite a second end.

16. The protection assembly of claim 15, wherein the end unit is extended away from the first end and past the second end when in the first state and retracted towards the first end when in the second state.

17. A protection assembly comprising:
a sleeve movable between a first sleeve position and a second sleeve position, wherein the sleeve covers an end unit of a robot when in the first sleeve position and does not cover the end unit when in the second sleeve position;
a biasing member configured to bias the sleeve to the first sleeve position; and
a locking mechanism configured to lock the biasing member in a locked state, wherein the biasing member does not bias the sleeve when in the locked state, and wherein when the locking mechanism releases the biasing member from the locked state, the biasing member transitions the sleeve from the second sleeve position to the first sleeve position.

18. The protection assembly of claim 17, wherein the end unit comprises a surgical tool or a surgical instrument.

19. The protection assembly of claim 17, wherein the biasing member comprises a spring.

20. The protection assembly of claim 17, further comprising a platform configured to receive the sleeve, wherein the sleeve is extended away from the platform and covers the end unit when in the first sleeve position and is positioned on the platform when in the second sleeve position.
